(19) United States
(12) Patent Application Publication
(10) Pub. No.: US 2022/0211417 A1
(43) Pub. Date: Jul. 7, 2022

(54) SYSTEM FOR MODIFYING A HUMAN OR ANIMAL BONE

(71) Applicants: Urs BRODBECK, Erlenbach (CH); Holger ZIPPRICH, Malchen (DE); Markus SCHLEE, Forchheim (DE)

(72) Inventor: Markus SCHLEE, Deutschland (DE)

(21) Appl. No.: 17/429,943

(22) PCT Filed: Feb. 11, 2020
(86) PCT No.: PCT/EP2020/053439

§ 371 Date: Aug. 11, 2021

(30) Foreign Application Priority Data
Feb. 12, 2019 (DE) 10 2019 201 825.4

(51) Int. Cl. A61B 17/66; A61C 8/02; A61F 2/28

(52) U.S. Cl. CPC A61B 17/666; A61C 8/0006; A61F 2002/30079; A61F 2002/2889; A61F 2/2846

(57) ABSTRACT

A system (1) for modifying a human or animal bone (2) is intended to reliably enable bone augmentation or bone reduction of the bone (2) to be treated in a particularly simple and, in particular, also particularly well-tolerated manner. For this purpose, according to the invention, a two-dimensionally extended lifting/pressure element (4) for insertion between the periosteum (6) of the bone (2) to be treated and the underlying bone (2) or the overlying connective tissue and a control element (10) associated therewith are provided, the control element (10) being designed to exert a magnetic pressure or tensile force on the lifting/pressure element (4).

The invention relates to a system for modifying a human or animal bone.

Particularly in the field of plastic or dental surgery, there may be a need for targeted modification of the existing bone. Both additive modifications, in which further bone mass is added to the existing bone mass, and subtractive modifications, in which the existing bone mass is reduced, may be desirable.

Additive modifications in the sense of bone augmentation may be necessary or desirable, particularly in the field of traumatology, plastic surgery or implantology, for example dental implantology, in order to be able to provide sufficient bone substance for secure anchoring of an implant in sufficient bone volume with sufficiently high primary stability. In fact, bone augmentation is usually necessary whenever the amount of existing bone is insufficient for implant placement or the jawbone structure is not right to firmly anchor the dental implant in the jaw. The reason for this can be, for example, bone loss or bone resorption that has occurred as a result of infection, toothlessness or dental disease and that continues to progress without adequate treatment. Then the rest of the dentition is also at risk of severe consequences for dental health and further tooth loss. Generally, in surgery, missing bone is built up or excess bone is removed; if this is necessary or desired. Bone augmentation may be required, for example, after an accident, tumor, tooth loss, or in cosmetic, or plastic surgery. Bone reduction can be useful, for example, in plastic surgery (nose, cheekbone correction, etc.) or in orthognathic surgery.

If bone augmentation should become necessary, the bone volume can be increased in the manner of a transplantation of autogenous bone by removing bone elsewhere and reinserting it at the recipient site. The disadvantage of such a method is that two operations have to be performed on the patient, which increases the risk of complications. Alternatively, the bone volume can also be increased by adding so-called bone substitute material. In this case, implants are usually made from bone substitute materials (human or animal donor bone or synthetic materials, especially alloplastic material) and inserted into the area to be augmented. However, allogeneic donor bone carries an immunological and/or infection risk. The available quantity of donor material is also limited for ethical and legal reasons. In the alternatively possible interspecies transfer of biomaterials (materials of animal origin, xenogeneic bone graft substitutes), there may be immunological problems and limitations arising from structural or anatomical differences. As a rule, implanted xenogenic bone substitute material is deposited at least for a long time in the bone regenerate or even only in the connective tissue. The biological potency for bone regeneration of such materials is limited.

Compared to autogenous bone grafts, the implantation of synthetic bone substitutes results in substitutes that must be regarded as incomplete substitutes without exception, especially in the case of ceramic implants. There is currently no ideal bone substitute.

The invention is therefore based on the task of specifying a system for modifying a human or animal bone, with which bone augmentation or bone reduction can be reliably achieved in a particularly simple and, in particular, particularly well-tolerated manner.

This task is solved according to the invention with a planar extended lifting/pressure element provided for insertion between the periosteum of the bone to be treated and the underlying bone or the overlying connective tissue, and with a control element associated therewith, wherein the control element is designed for exerting a magnetic pressure or tensile force on the lifting/pressure element.

The invention is based on the consideration that for a particularly well-tolerated and also reliable modification of bone volume, recourse should be made to the natural mechanisms of bone formation and growth. The natural formation of bone, or remodeling, or healing of a bone injury, occurs through osteoblasts. These bone-forming cells and their precursors are found in the periosteum (periosteum), around blood vessels (pericytes), in the inner periosteum (endost), and in the bone itself. In expanded bone tissue, for example in the case of fissure formation as a result of bone fractures, pericytes migrate from the vessel walls of the expanded tissue and develop into osteoprogenitor cells and later into osteoblasts. Vascular anastomoses from the bone (Volkmann's vessels) are connected to vessels in the stratum fibrosum of the periosteum. The osteoprogenitor cells or osteoblasts in the cambium of the periosteum are also involved in the synthesis of new bone. Therefore, deformations of the periosteum, especially gradual distraction from the bone surface, lead to new bone formation (bone deposition) on an existing bone surface.

Using this knowledge in a targeted manner, it is now intended to create a free space above the bone and below the periosteum or periosteum in a targeted and adjustable manner for the purpose of bone formation, into which the percytes can migrate and subsequently cause new bone formation. In the past, metal plates or grids were inserted to create the free space. The soft tissue was mechanically lifted from the bone by metal pieces perforating the soft tissue. This procedure carries a risk of infection due to the perforation, and the desired shape of the bone can only be insufficiently regenerated. In contrast, a flat lifting or pressure element is now provided in accordance with the invention to create said free space, which is inserted between the bone and periosteum for the lifting movement and then lifts the periosteum relative to the bone by the application of a force from the outside. In this way, the said free space is formed in a targeted manner and bone formation can take place.

In the opposite case, i.e. when subtractive modification with targeted bone resorption is desired, a force can be applied to the pressure element in the opposite direction, i.e. towards the bone, which exerts pressure on the periosteum. In this case, the pressure element is inserted between the periosteum and the surrounding or overlying connective tissue. In response to this pressure, the bone substance recedes so that the desired local reduction in bone volume is achieved.

In order to achieve this two-dimensional application of force via the lifting/pressure element in a particularly gentle manner and in a manner that is compatible with the patient, the application of force to the lifting/pressure element by an associated external control unit is designed in a contactless manner, particularly preferably magnetically. The control unit can comprise a suitably selected magnet and/or the lifting/pressure element can be suitably magnetically designed so that the desired force-side coupling is achieved by the magnetic interaction. The force-side coupling is achieved in particular by the magnetic field generated by the control unit acting on the magnetization of the stroke/pressure element and/or its magnetic moment and thus generating the desired magnetic force.

Advantageous embodiments of the invention are the subject of the dependent claims.

For the purpose of bone augmentation, the lifting/pressure element is advantageously implanted temporarily between bone and periosteum or between periosteum and connective tissue. With regard to its properties, such as flexibility, thickness, permeability or the like, the lifting/pressure element is advantageously designed in such a way that adequate permeability enables reanastomosis of the periosteum with vessels in the bone. On the other hand, the lifting/pressure element should be sufficiently mechanically stable to be able to transmit forces to the periosteum. It should also be as thin as possible to facilitate coverage with the skin flap and to make surgery easier during insertion. After adequate healing time, the lifting/pressure element is increasingly separated from the bone by constant or periodically acting forces, for example magnetic forces. This results in the deposition and formation of new bone. By controlling the direction of pull of the forces, their intensity and duration, the shape of the bone can be predetermined or -influenced.

For the purpose of bone resorption, the pressure element is temporarily implanted between the periosteum and the overlying connective tissue. After an adequate healing time, the pressure element exerts pressure on the periosteum and bone through controlled forces. This results in bone resorption. By controlling the direction of pressure of—the forces, their intensity and duration, the shape of the bone can be predetermined.

Particularly preferably, the lifting/pressure element is designed with the following criteria and design goals in mind:
1. It should be as thin as possible. This ensures that after exposure of the bone and application of the lifting/pressure element, the periosteum and the overlying soft tissue can be closed over the lifting/pressure element without tension. This is—important for wound healing. On the other hand, there is a limit here but also—sufficient mechanical stability to be able to transmit forces.
2. It should be flexible, i.e. changeable in shape, in order to be able to change from “small to large” when bone is built up and analogously vice versa when bone is—reduced.
3. It should preferably be perforated or otherwise permeable in surface area to allow vascular reconnection between vessels in the bone and periosteum. Advantageously, a ratio of perforation to surface area of at least 10%, particularly preferably at least 30%, is provided. The pore size of the pores forming the perforation is preferably selected small enough to prevent “slipping through” of the periosteum and to achieve uniform elevation of the entire soft tissue.

The associated control element, on the other hand, is preferably suitably designed with regard to the intended magnetic force transmission for generating a magnetic field in the region of the intended bone modification. For this purpose, the control element may comprise a number of suitably selected and positioned permanent magnets and/or a suitably controllable electromagnet. The control element is preferably designed for positioning outside the patient's body is provided and designed; for example, it could be attachable to the outside of the patient's face in the jaw region by a facemask, strap, splint or the like in the case of intended modification of a jaw bone.

The system, comprising the lifting/pressure element and the associated control element, is preferably designed with regard to the magnetic coupling and force transmission-suitable for the desired creation of the free space between periosteum and bone or between periosteum and connective tissue. For this purpose, the force transmitted magnetically from the control element to the lifting/pressure element should be selected approximately such that per day the resulting bone distraction is about 0.5 mm. To make this possible, the lifting/pressure element advantageously has a suitably selected magnetization or magnetizability for this purpose.

The lifting or pressure element can be manufactured in an expandable manner, for example as a grid, knitted, woven or crocheted fabric. The associated second magnetic-part or control element is preferably mounted, for example, in a splint that maps the desired shape of the bone. This enables targeted, guided regeneration of natural bone to the desired volume.

Particularly preferably, the lifting/pressure element is permanent- or ferromagnetic.

The lifting/pressure element can have a substantially constant magnetization or magnetizability when viewed over its surface. However, in order to enable a new vessel connection between the vessels in the bone and the periosteum and thus to particularly promote the formation of new bone, the lifting/pressure element is particularly preferably perforated or otherwise permeable in its surface. In conjunction with this, the lifting/pressure element advantageously also has a magnetization or magnetizability dis-tributed over its surface in the manner of a grid structure.

To provide the intended magnetic or magnetizable properties, the lifting/pressure element preferably has components made of a suitably selected material, preferably a magnetic or magnetizable metal, particularly preferably iron, in a suitable form, for example as wire, flakes, platelets or particles. In the sense of particularly good biocompatibility or compatibility, these components are preferably suitably surrounded by and completely embedded in a biocompatible material, in particular a plastic, in the manner of an encapsulated design. This can be achieved in a particularly simple manner and also in a particularly user-friendly and flexible manner by the lifting/pressure element being formed in a very particularly preferred embodiment by a plastic matrix in which magnetic or magnetizable particles are embedded. Particularly preferred materials for this are Teflon, polypropylene or other materials from which threads are made for surgi-cal applications.

The advantages achieved with the invention consist in particular in the fact that the—lifting/pressure element, which can be externally applied with a suitable force and is—intended for insertion between the periosteum and the bone or between the periosteum and the connective tissue, can achieve a modification of the bone to be treated in a particularly gentle manner that comes close to the natural mechanisms. This means that the desired bone modification can be achieved in a particularly gentle manner that is particularly well tolerated by the patient.

An embodiment of the invention is explained in more detail with reference to a drawing. Therein show:

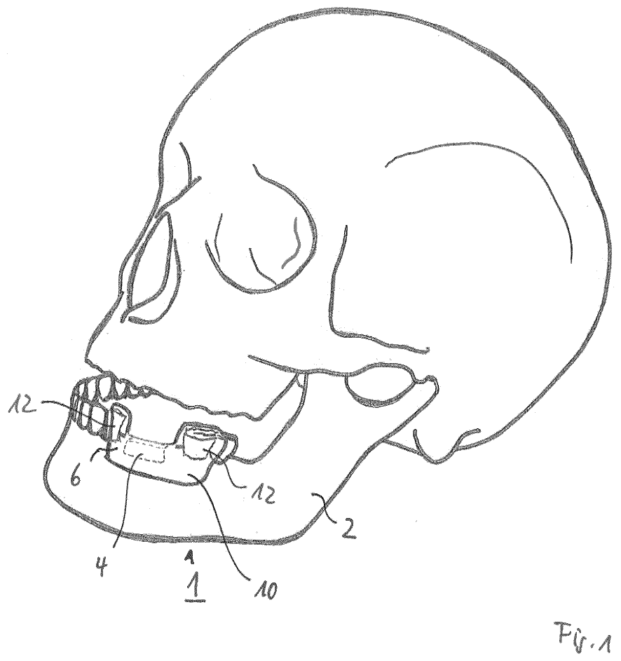

FIG. 1 a system for modifying a human or animal bone,

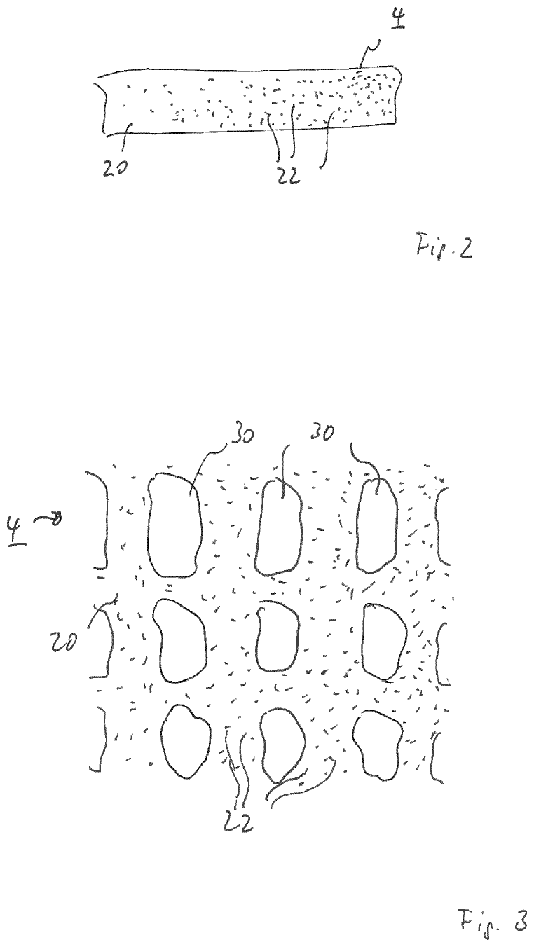

FIG. 2 a lifting/pressure element of the system according to FIG. 1 in sectional cross section, and

FIG. 3 the lifting/pressure element according to FIG. 2 in plan view.

Identical parts are marked with the same reference signs in all figures.

The system 1 according to FIG. 1 is designed for the targeted modification of a human or animal bone in a particularly gentle manner that is compatible with the patient. In the embodiment example according to FIG. 1, this is shown for the treatment of the jaw bone 2 of a patient; however, other embodiments, directed at the treatment of other bones 2 of humans or animals, are of course also included in accordance with the invention.

The system 1 is designed for an operating principle, which is in itself considered to be in accordance with the invention, for achieving a desired bone modification, in which a lifting/pressure element 4 extended over an area is positioned in or on the bone 2 and then subjected to a suitably selected force. In the case of an intended bone augmentation, the lifting/pressure element 4 is positioned between the periosteum or the periosteum 6 and the actual bone 2. In this case, the external force applied to the lifting/pressure element 4 lifts the periosteum 6 away from the actual bone 2, creating a free space between the bone 2 and the periosteum 6. Percytes can migrate into this free space from the vessel walls of the expanded tissue, which subsequently cause new bone formation in the free space created. In the other case, namely when bone resorption is desired, the lifting/pressure element 4 is positioned between the periosteum 6 and the surrounding connective tissue and a force is applied in the direction of the bone 2 so that pressure is exerted on the bone 2. This causes the bone 2 to recede and thus the desired bone resorption.

To transmit or initiate the intended force to/into the lifting/pressure element 4, a control element 10 is assigned to the latter, which is suitably coupled or can be coupled to the lifting/pressure element 4 on the force side. In this case, since the embodiment example involves the intended treatment of a jaw bone 2 in the context of dental surgery, the control element 10 is designed as a splint which is suitably placed on the gum and fastened at the end to the teeth 12 of the patient. Alternatively, the control element 10 could be positioned outside the patient and attached to the patient's head, for example, by means of a harness system, a suitable facemask, or any other attachment system.

In the embodiment, magnetic force transmission is provided between the control element 10 and the lift/pressure element 4. The rail forming the control element 10 is suitably equipped to generate a magnetic field in the intended positioning area of the lifting/pressure element 4 at or on the jawbone 2. For this purpose, the control element 10 may comprise a number of suitably selected selectively actuatable electromagnets; in the embodiment example, however, the control element 10 is equipped with a number of permanent magnets. When equipped with electromagnets, the magnetic field in the—area of the positioning of the lifting/pressure element 4 and thus the force acting on the lifting/pressure element 4 and transmitted by it to the periosteum 6 can be suitably adjusted in a particularly simple manner by electrical control of the control element 10 and readjusted as required, i.e., for example, readjusted in accordance with a determined treatment success.

The system 1, comprising the lifting/pressure element 4 and the associated control element 10, is suitably designed in terms of magnetic coupling and force transmission for the desired creation of the free space between periosteum 6 and bone 2 or between periosteum 6 and connective tissue. For this purpose, the force transmitted magnetically from the control element 10 to the lifting/pressure element should be selected approximately such that per day the resulting bone distraction is approximately 0.5 mm. In the embodiment example, the lifting/pressure element 4 is based on encapsulated or Teflon-sheathed esiens and is thus ferromagnetic. The magnetization or magnetizability of the lifting/pressure element 4 is selected appropriately with regard to the force to be transmitted.

To carry out the intended treatment, the lifting/pressure element 4 is temporarily implanted between bone 2 and periosteum 6 or between periosteum 6 and connective tissue. With regard to its properties, such as flexibility, thickness, permeability or the like, the lifting/pressure element 4 is designed in such a way that, on the one hand, the desired placement is particularly easy and, on the other hand, adequate permeability enables reanastomosis of the periosteum 6 with vessels in the bone 2.

In particular, the lifting/pressure element 4 is designed with a particularly low thickness, i.e. as thin as possible. This ensures that during implantation, after exposure of the bone 2 and application of the lifting/pressure element 4, the periosteum 6 and the overlying soft tissue can be closed over the lifting/pressure element 4 without tension. This is important for wound healing. On the other hand, the thickness is also chosen to be sufficiently large so that sufficient mechanical stability is ensured to transmit the intended forces.

In order to be able to change appropriately during treatment, i.e. from “small to large” in the case of bone augmentation or vice versa in the case of bone resorption, and to adapt to the changing environmental conditions in the course of treatment, the lifting/pressure element 4 is designed to be sufficiently flexible, i.e. changeable in shape. In order to enable equally reliable magnetizability over an extended area, the lifting/pressure element 4, as shown in the enlarged cross-sectional view in FIG. 2, is formed by a base body 20 made of a suitably selected, in particular biocompatible and also sufficiently flexible plastic. The base body 20 thereby forms a plastic matrix in which magnetic or magnetizable particles 22 are embedded. Due to this structure, the particles 22 are completely embedded in the plastic base body 20 in the manner of an—encapsulated design in the sense of particularly good biocompatibility or compatibility. To provide the desired magnetic properties, the particles 22 thereby consist of iron.

In order to enable a new vessel connection between vessels in the bone 2 and in the periosteum 6 during treatment, i.e. even with the lifting/pressure element 4 implanted,—the lifting/pressure element 4 is perforated and thus designed to be permeable on the surface. This can be seen particularly well in the top view of the lifting/pressure element 4 according to FIG. 3. In order to form the perforation, the base body 20 forming the lifting/pressure element 4 is provided with holes 30, which are kept comparatively large, when viewed from the surface, with the base body 20 forming a kind of grid-net body—between them. Alternatively, smaller holes could of course be provided to form the—desired perforation. In the embodiment example, a perforation to area ratio of about 50% is provided. Moreover, the pore size of the holes 30 forming the perforation, which is defined by the size of the holes 30, is selected small enough to prevent the periosteum 6 from “slipping through” and to achieve uniform elevation of the entire soft tissue.

Due to the shape of the base body 20 on the one hand and the basic structure as a matrix body with embedded magnetic particles 22 on the other hand, it results for the embodiment example shown that the stroke/pressure element 4 shown has a—magnetization or magnetizability distributed over its surface in the manner of a grid structure.

### LIST OF REFERENCE SIGNS

1 System
2 Bone
4 Lift/pressure element
6 Periost
10 Control
12 Tooth
20 Base Body
22 Particle
30 Hole

1-5. (canceled)

6. System (1) for modifying a human or animal bone (2), having a lifting/pressure element (4) which is provided for insertion between the periosteum (6) of the bone (2) to be treated and the underlying bone (2) or the overlying connective tissue and which is formed by a plastic matrix in which magnetic or magnetisable particles (22) are embedded, and having a control element (10) associated therewith, the control element (10) being designed to exert on the lifting/pressure element (4) a magnetic pressure or tensile force which is selected in such a way that the resulting bone distraction per day, taking into account the magnetisation of the lifting/pressure element (4), is approximately 0.5 mm.

7. System (1) according to claim 6, the lifting/pressure element (4) of which is permanent or ferromagnetic.

8. System (1) according to claim 6, the lifting/pressure element (4) of which has a substantially constant magnetisation or magnetisability seen over its surface.

9. System (1) according to claim 6, the lifting/pressure element (4) of which has a magnetisation or magnetisability distributed over its surface in the manner of a lattice structure.
